(19) United States
(12) Patent Application Publication
(10) Pub. No.: US 2016/0130574 A1
(43) Pub. Date: May 12, 2016

(54) METHODS OF MEASURING GENE EXPRESSION IN FACS-SORTED CELLS

(71) Applicant: Merck Sharp & Dohme Corp., Rahway, NJ (US)

(72) Inventors: Svetlana Sadekova, Mountain View, CA (US); Robert H. Pierce, San Francisco, CA (US); Bela Desai, Saratoga, CA (US)

(73) Assignee: Merck Sharp & Dohme Corp., Rahway, NJ (US)

(21) Appl. No.: 14/243,451

(22) Filed: Apr. 2, 2014

Related U.S. Application Data
(63) Continuation of application No. 12/970,770, filed on Dec. 16, 2010, now abandoned.
(60) Provisional application No. 61/289,514, filed on Dec. 23, 2009.
(60) Provisional application No. 61/379,878, filed on Sep. 3, 2010.

(51) Int. Cl. C12N 15/10; C12Q 1/68; G01N 33/569

(52) U.S. Cl. CPC C12N 15/1003; G01N 33/56972; C12Q 1/686

(57) ABSTRACT

Improved methods of measuring gene expression in intracellularly immunostained FACS-sorted cell populations are provided. Exemplary methods involve fixing cells with formalin and permeabilizing with mild detergent in the presence of ribonucleoside vanadyl complex prior to FACS sorting, followed by RNA extraction in the presence of de-crosslinking agents. The resulting RNA is suitable for gene expression analysis. The method allows for analysis of the gene expression pattern specifically associated with any sortable cell population or subpopulation.

This application is a continuation of U.S. patent application Ser. No. 12/970,770, filed Dec. 16, 2010, which claims priority to U.S. Provisional Patent Application Nos. 61/289,514 and 61/379,878, filed Dec. 23, 2009 and Sep. 3, 2010, respectively, the disclosures of which are hereby incorporated by reference in their entireties.

### FIELD OF THE INVENTION

The present invention relates generally to methods of measuring gene expression in cells.

### BACKGROUND OF THE INVENTION

Methods of determining gene expression profiles have advanced significantly in recent years. Gene expression profiles are useful in determining what genes are involved in various biological responses, such as disease-related responses. Genes with altered expression in disease may represent new targets for therapeutic intervention, or disease diagnosis, prognosis and monitoring. Gene expression profiles are also useful in determining which genetic pathways, cells and cell-types are involved in disease pathology.

Gene expression is often performed on heterogeneous populations of cells. Any resulting gene expression pattern will thus represent the superposition of the patterns for all cells in the population, which may mask specific gene expression differences associated with any given sub-population of the cells.

Accordingly, the need exists for improved methods for measuring gene expression in selected relatively homogenous subsets of cells, such as cells sorted by fluorescence activated cell sorting (FACS). Preferably, such methods would permit isolation of intact mRNA suitable for gene expression analysis from cells that have been FACS sorted based on intracellular staining.

### SUMMARY OF THE INVENTION

The present invention meets these needs and more by providing improved methods of obtaining mRNA from subsets of cells within a larger, more heterogeneous population. Specifically, the methods involve obtaining mRNA from cells that have previously been sorted by fluorescence-activated cell sorting (FACS) using intracellular staining. Such populations can be essentially homogenous with respect to the characteristics upon which they were sorted. The pattern of gene expression from such sorted cells can thus be conclusively ascribed to the sorted cell subpopulation. Such methods also potentially allow detection of differences in gene expression in small (sorted) subsets of cell population that would otherwise go unnoticed (i.e. not rise to statistical significance) in the presence of excess mRNA from contaminating non-sorted cells. Accordingly, the methods of the present invention provide for gene expression analysis with increased specificity and sensitivity.

In some embodiments, FACS sorting is performed based on intracellular protein staining. Intracellular staining may be against any cytoplasmic or nuclear antigen, such as proteins involved with progression through the cell cycle. Cytoplasmic proteins may also encompass proteins that would otherwise be secreted, such as cytokines, if a protein translocation inhibitor is used to prevent secretion. In some embodiments, intracellular staining is effected using a fluorescently labeled antibody.

In one embodiment, a population of cells is treated with an RNase inhibitor (e.g. ribonucleoside vanadyl complex, or RVC), fixed with formalin, and permeabilized with mild detergent prior to FACS sorting. In some embodiments, RVC is added to cells before, concurrently with, or very shortly after fixation/permeabilization so that it is present and able to prevent RNA degradation through the intracellular staining and cell sorting steps, and is also added to the sorted cells. In various embodiments of the present invention, RVC is added to a concentration during fixation of 50 mM, 20 mM, 10 mM, 5 mM, 2 mM, 1 mM, 0.5 mM or lower. In selected embodiments, RVC is added to a concentration of 10 mM, 5 mM, or 2 mM. In one embodiment, RVC is not included in the buffer used to resuspend the cells for intracellular staining.

In some embodiments the fixation and permeabilization steps are performed using the Cytofix/Cytoperm Fixation and Permeabilization Kit (BD Biosciences, San Jose, Calif., USA). In some embodiments formalin fixation is performed using a 4% paraformaldehyde solution. In some embodiments, the mild detergent used to permeabilize the cells is saponin, e.g. 0.1% saponin.

In various embodiments, the mRNA is then extracted from the sorted cells in a process involving use of a de-crosslinking agent. In various embodiments, RNA extraction is performed using FFPE RNA Extraction Kit (Roche Applied Science, Indianapolis, Ind., USA). In some embodiments, the de-crosslinking agent is proteinase-K.

Gene expression profiles from FACS-sorted cell populations will find use in a variety of research and clinical contexts. In research, cells can be sorted based on cytokine expression and then subjected to gene expression analysis to detect what genes are associated with the selected cytokine expression phenotype. In the clinic, cells from human subject can be sorted to isolate cell subsets of interest to perform gene expression analysis to detect abnormal gene expression at any given point in time (e.g. for diagnosing disease or in assessing propensity or prognosis for disease) or monitor changes in gene expression over time (e.g. to monitor gene expression and/or response to therapy). Therapeutic intervention may then be altered (if necessary) in response to such gene expression results. In various embodiments, gene expression patterns determined using methods of the present invention are used to determine disease propensity, predict disease, diagnose disease, or monitor disease progression. In other embodiments, gene expression patterns determined using methods of the present invention are used to track disease biomarkers to evaluate target engagement, determine whether a drug is efficacious in a clinical trial, determine efficacy of a given therapeutic approach in a given subject, or modify (if necessary) a therapeutic regimen based on the subject's response. Modification of therapeutic regimen can include modification of dosage and/or dosing interval, including discontinuance of therapy altogether.

### BRIEF DESCRIPTION OF THE DRAWINGS

FIG. 1 shows the results of experiments in which RNA was isolated from CD4⁺ peripheral blood mononuclear cells (PBMCs) that had been sorted based on IL-17 production, using either a control RNA isolation procedure or the procedure of the present invention (referred to as the “SPB protocol”). A control RNA sample is shown in the leftmost lane, along with RNA isolated by the SPB protocol from IL17⁺ or IL17⁻ cells. TaqMan® gene expression analysis results using RNA obtained from these IL17⁺ and IL17⁻ cell populations showed 645-fold higher IL17 gene expression in the IL17⁺ population.

FIGS. 2A and 2B show sorts of human CD4⁺ T cells that were polarized either under Th17 conditions (FIG. 2A) or Th1conditions (FIG. 2B) and sorted based on IL-17 and IFNγ levels, as measured by intracellular immunostaining. Data for one donor are shown and are representative of at least eight independent experiments. Cells were pooled into six separate populations based on polarization conditions and cytokine expression: Th17 (IL17⁺ IFNγ⁺) (population D, 19.9% of sort); Th17 (IL17⁺ IFNγ⁻) (population A, 14.2% of sort); Th17 (IL17⁻ IFNγ⁺) (population E, 35.7% of sort); Th17 (IL17⁻ IFNγ⁻) (population B, 30.2% of sort); Th1 (IFNγ) (population C, 66.4% of sort) and Th1 (IFNγ⁺) (population F, 33.5% of sort). Th1 cells were IL17⁻.

FIG. 3A is a plot of IL-17 and IFNγ gene expression, on a log/log scale, in the various subpopulations of cells obtained in the sort shown in FIG. 2. Datapoints represent cells from different donors. Boxes are provided grouping the datapoints for each sort subpopulation (A-E).

FIG. 3B shows IL-17A expression, expressed as mean fluorescence intensity (MFI), in Th17 and Th1 polarized cells that sort as IL17⁻. Lines connect Th17- and Th1-polarized samples from the same donor.

FIGS. 4A-4D show expression of the IL17F, IL22, CCL20 and IL26 genes in the various subpopulations A-E obtained in the sorts illustrated in FIG. 2. One asterisk (*) indicates statistically significant differences (P<0.05) between IL17⁻/IFNγ⁺ Th17 cells and IL17⁺/IFNγ⁺ Th1 cells. Two asterisks (**) indicates P<0.01.

### DETAILED DESCRIPTION

All references cited herein are incorporated by reference to the same extent as if each individual publication, database entry (e.g. GenBank sequences or GeneID entries), patent application, or patent, was specifically and individually indicated to be incorporated by reference. Incorporation by reference of any patent or published patent application is intended to incorporate the sequences in the sequence listing for that patent or published patent application.

As used herein, including the appended claims, the singular forms of words such as “a,” “an,” and “the,” include their corresponding plural references unless the context clearly dictates otherwise. As used herein, unless otherwise indicated, recited concentrations refer to the final working concentration for use in the experiment, i.e. the concentration of the agent at the time the experiment is performed.

Although the methods of the present invention may be conveniently described as a list of steps, such steps need not necessarily be performed in any particular order unless such order is logically required. For example, steps involving addition of the RNase inhibitor RVC, fixation of cells, and permeabilization of cells can be performed in any order (or effectively simultaneously), as would be understood by one of skill in the art, provided that RVC is present at the appropriate time to protect RNA from degradation. Specifically, it is intended that RVC be added before, simultaneously with, or very shortly after the fixation/permeabilization steps. Other steps, however, will logically require sequential performance, such as RNA extraction from sorted cells, which must necessarily be performed after cells are sorted.

### I. Determining Gene Expression Profiles in FACS Sorted Cells

Prior art methods of fixing cells prior to FACS, such as acetone fixation, typically lead to low quality mRNA inadequate for use in gene expression analysis. Not all methods of inhibition of RNase support production of mRNA of sufficient quality for gene expression analysis. For example, RNasin® was found to be inferior for use in the methods of the present invention. RNAlater® (Ambion, Inc., Austin, Tex., USA), a commercial ammonium sulfate composition to enhance stability of RNA at room temperature, is also described for use in purification of RNA from FACS sorted cells. Barrett et al. (2002) BioTechniques 32:888; U.S. Pat. No. 6,204,375.

Ribonucleoside-vanadyl complexes (RVCs) are stable cyclic 2′,3′-monophosphate transition state analogs for ribonucleases that have been long been proposed for use in protecting RNA from degradation. Puskas et al. (1982) Biochemistry 21:4602. RVC has also been used as an RNase inhibitor during RNA preparation from ethanol-fixed and FACS sorted cells. Esser et al. (1995) Cytometry 21:382. Methods have also been described for preservation of RNA in immunohistochemical (IHC) sections, such as samples excised using Laser Capture Microdissection (LCM), using RVC. See U.S. Pat. App. Pub. No. 2004/0265840. In addition, Pechhold and co-workers describe the use of RVCs (15 mM) in a method of measuring gene expression in FACS sorted cell populations using a quantitative nuclease protection assay (qNPA). Pechhold et al. (2009) Nat. Biotech. 27:1038.

In selected embodiments of the present invention, the concentration of RVC is kept low to improve sorting yield. For example, a range of RVC concentrations (e.g. 1 mM to 10 mM) may be used during fixation in parallel experiments, and the concentration of RVC giving the best sorting yield while maintaining adequate mRNA quality is chosen for gene expression analysis experiments. In some embodiments, RVC is used at a final working concentration of 2 mM or 5 mM during fixation.

In one embodiment the method of the present invention involves one or more washing steps after fixation and permeabilization, but before intracellular staining (e.g. immunostaining), wherein the cells are resuspended after the washing using a buffer than does not contain RVC. Note that the resuspended cells may contain some residual RVC despite washing steps and resuspension using a buffer without RVC.

In embodiments of the present invention, RVC is present during fixing/permeabilization, intracellular staining, and sorting. Cells to be sorted can be from a preparation of primary cells, from cell culture, from a tissue, from a tumor, from immunohistochemically defined sections (or LCM-extracted portion thereof), etc. Cells can be sorted based on the presence or absence of any protein normally within the cell, such as nuclear proteins or cell cycle proteins, or even proteins that are usually secreted. To detect a protein that is usually secreted, a protein transport inhibitor, such as GolgiStop (monensin) or brefeldin A, is used to ensure the cytokines accumulate within the cell (e.g. the Golgi) for detection by intracellular staining. For example, cells can be stained for cytokine production with fluorochrome-conjugated anti-cytokine antibodies. Intracellular cytokine staining can be supplemented with staining for cell surface markers to further specify the cell-type to be sorted. Staining for cell surface markers is typically performed before fixation and permeabilization of the cells.

In one embodiment, the present invention provides methods of sorting T cells into Th17 and Th1 subsets based on cytokine production, as measured by intracellular staining of cells treated with a protein translocation inhibitor, and optionally, staining of cell surface markers specific for Th17 and/or Th1 cells.

In another embodiment, the intracellular staining involves immunofluorescent staining for cell cycle markers, such as phospho-Histone 3, to identify cells in M phase, and/or Ki 67, which is present in all cycling cells, but not in quiescent (G0) cells. When cells are grown in the presence of bromodeoxyuridince (BrdU), e.g. at 20 μM during the final hour of culture, a fluorescent anti-BrdU antibody may be used to determine the frequency of cells that had synthesized DNA, i.e. cells in S-phase. In exemplary embodiments the antibodies to phospho-Histone 3, Ki 67 and BrdU are labeled with Alexa Fluor® 647, Alexa Fluor® 555, and Alexa Fluor® 488, respectively.

Although the methods of the present invention are of particular value in preserving RNA for gene expression analysis in cells that have been sorted based in intracellular immunofluorescence staining, the methods will be generally useful in methods requiring isolation of intact mRNA under conditions that would otherwise result in RNA degradation.

### II. Examples

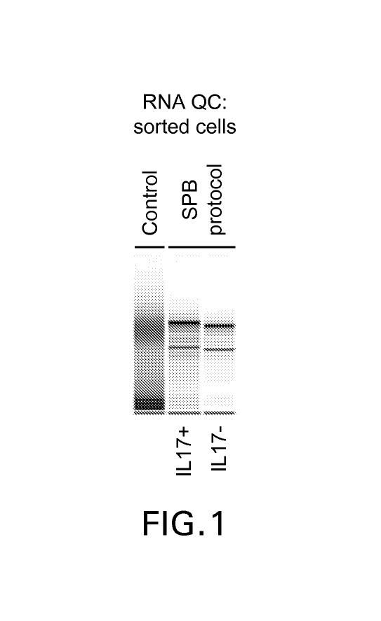

Exemplary protocols of the present invention are provided at Examples 2 and 3, and results are provided at FIGS. 1-4. One of skill in the art would recognize that the methods of the present invention need not be performed exactly as described in the Examples, and that most steps can be modified without deviating from the claimed invention.

Messenger RNA prepared using the methods of the present invention (Example 2) is significantly cleaner that RNA prepared using prior art methods, and shows far less degradation (smearing) when analyzed by gel electrophoresis. FIG. 1A

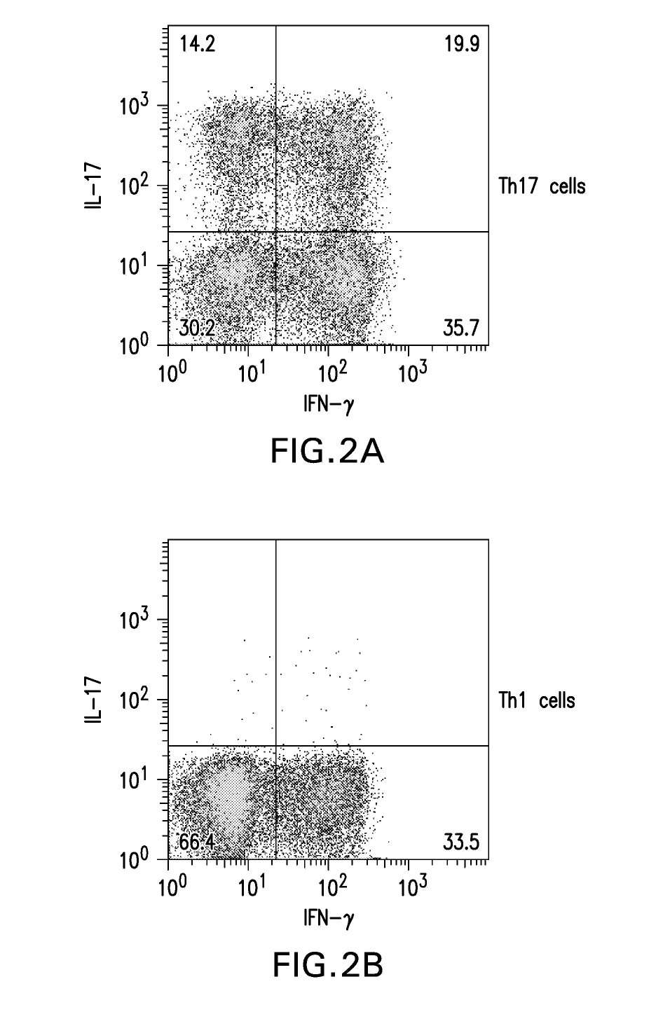

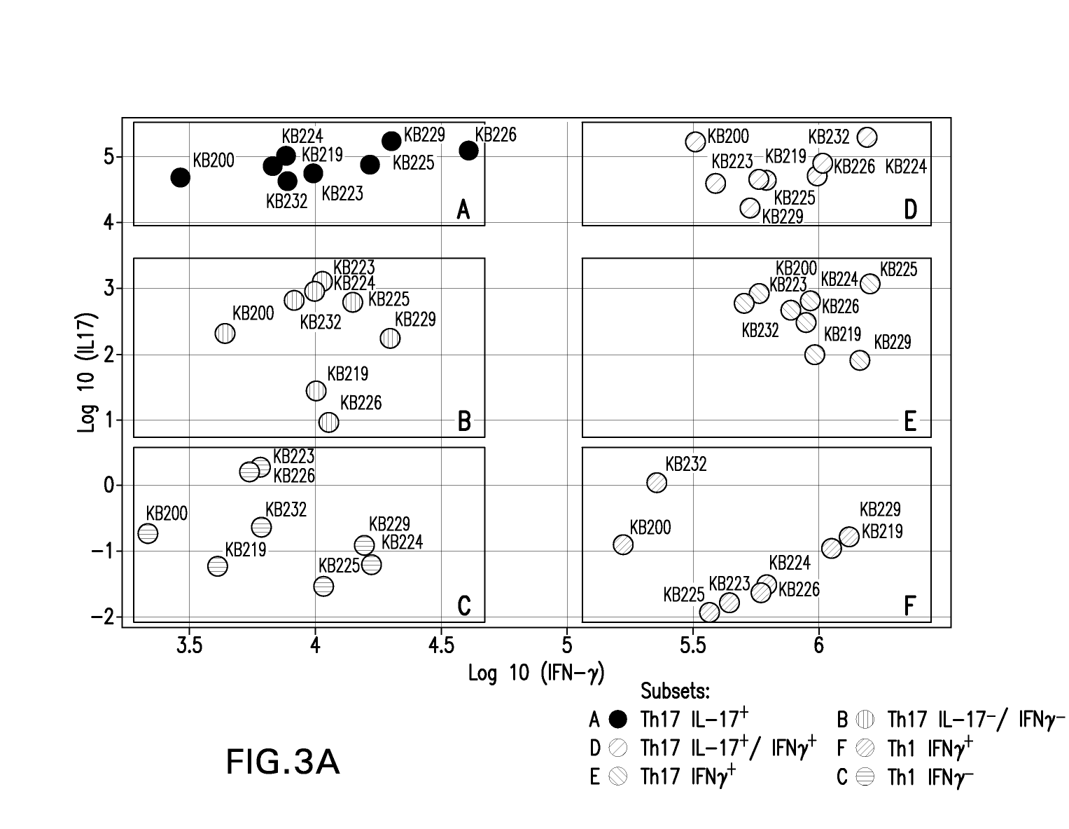

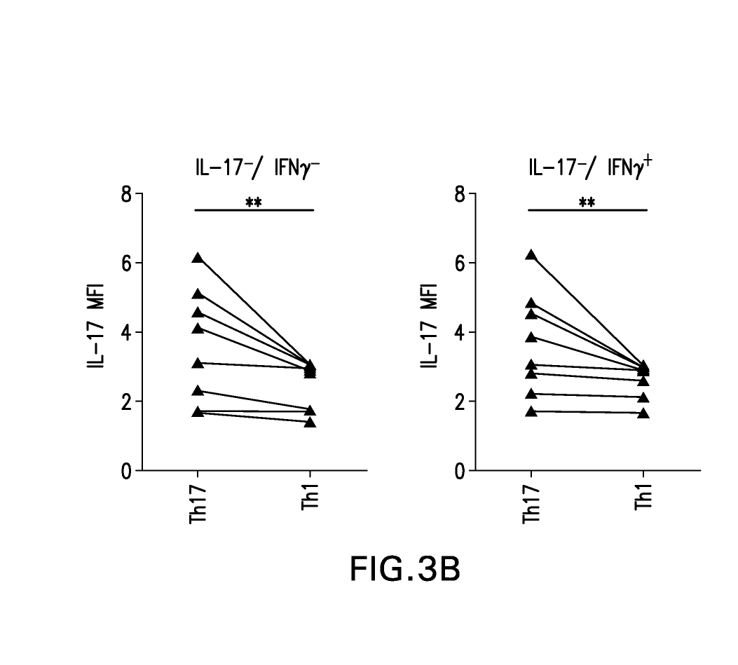

Example 3 illustrates one application of the methods of the present invention. In both healthy people and Crohn's disease patients, CD4⁺ T cells can be identified that express interleukin 17 (IL-17), interferon gamma (IFNγ), or both (“double positive” T cells). Because Th1 cells are known to be IFNγ⁺, and Th17 are typically characterized as being IL-17⁺, it was of interest to determine the characteristics of the observed double positive T cells. As illustrated in greater detail in Examples 2 and 3, human cells were cultured in vitro under Th1- or Th17-polarizing conditions, and then stained for intracellular accumulation of IL-17 and IFNγ. Treatment with GolgiStop, a protein transport inhibitor, caused cytokines that would otherwise be secreted to accumulate within the cells. Both Th1 and Th17 cell populations were then sorted based on intracellular IL-17 and IFNγ levels. The results show that Th1-polarized cells do not produce IL-17, but that a substantial portion of Th17-polarized cells produce IFNγ. FIGS. 2A and 2B. Gene expression analysis of these sorted cells confirmed that the expression of IL-17 and IFNγ mRNA correlated with protein expression (which was the basis for the sort), and that the sorts were reproducible in that the gene expression data for all individuals within a given sort clustered together. FIG. 3A. As shown in FIG. 3B, even Th17 cells that sort as IL17⁻ express significantly higher levels of IL17 than IL17⁻ Th1 cells.

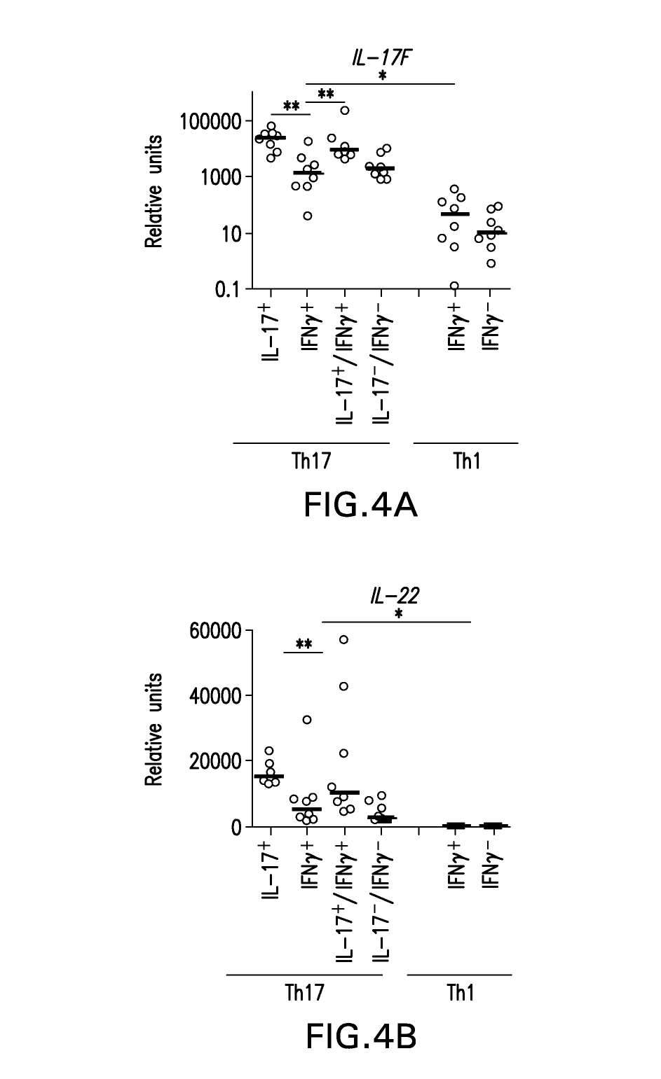

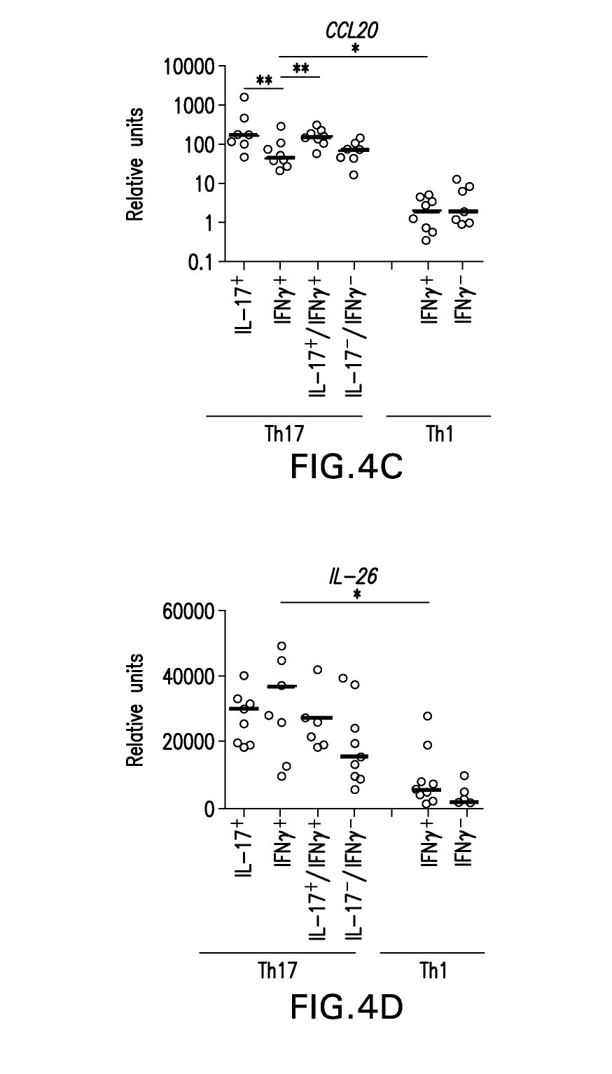

Further gene expression analysis of the six sorted populations of cells, as shown in FIGS. 4A-4D, shows that even though they share the same sort phenotype, IL17⁻/IFNγ⁺ cells polarized under Th17 conditions show uniformly higher levels of several Th17-associated proteins, i.e. IL-17F, IL-22, CCL20 and IL-26 (Wilson et al. (2007) Nature Immunol. 8:950), compared with IL17⁻/IFNγ⁺ cells polarized under Th1 conditions.

The results as a whole demonstrate that Th17 IFNγ producers and Th1 IFNγ producers represent distinct cell populations exhibiting different cytokine expression profiles, highlighting the need to further analyze IFNγ⁺ (single positive) cells before assuming they are typical Th1 cells. These results may have practical significance in driving further research into mechanism of inflammatory disease, and therefore its treatment.

The broad scope of this invention is best understood with reference to the following examples, which are not intended to limit the inventions to the specific embodiments therein. One of skill in the art would recognize that different cell types (e.g. other than human PBMCs) may require slightly different conditions, such as concentration of RVC, time of permeabilization or concentration permeabilizing detergent, for optimal gene expression analysis. Such routine optimization is within the skill in the art, and would not constitute an undue burden in such types of experiments.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd}ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalog, Molecular Probes, Inc., Eugene, Oreg.; Sigma-Aldrich (2003) Catalog, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila., Pa.; Louis, et al. (2002) Basic Histology: Text and Atlas, McGraw-Hill, New York, N.Y.

Statistical analysis may be performed using commercially available software, including but not limited to JMP® Statistical Discovery Software, SAS Institute Inc., Cary, N.C., USA.

Cell growth media and methods are provided, e.g., at Int'l. Pat. Appl. Pub. No. WO 90/03430 and U.S. Pat. No. 5,830,761, the disclosures of which are hereby incorporated by reference in their entireties.

### Example 2

### Gene Expression Analysis of Human PBMC Sorted on IL-17 and IFNγ

Messenger RNA is obtained from human peripheral blood mononuclear cells (PBMC) sorted for expression of both interleukin 17 (IL-17) and IFNγ and used for gene expression analysis, as follows. All steps after cell activation are performed at 4° C. or on ice. All solutions and reagents are also pre-cooled at 4° C. or on ice. All solutions used in the fixing step and thereafter are prepared using nuclease free, DEPC treated water. Cells to be analyzed for gene expression are cultured in a CO₂ incubator set at 5%, and treated as required to induce the desired differentiation. See Example 3.

### Fixation and Permeabilization

Cells are washed with Yssel's medium plus 1% human AB serum (Cat. No. 400-103, Gemini Bio-Products, Woodland, Calif., USA), and counted using a Vi-CELL™ Series Cell Viability Analyzer (Beckman Coulter, Inc., Brea, Calif., USA). In some embodiments, 50 μg/ml gentamicin is included in the wash.

Cells are then activated for 4.5 hours at 37° C., at 10⁶ cells/ml, in the presence of 500-1000 ng/ml ionomycin, 25 ng/ml phorbol 12-myristate 13-acetate (PMA), and the protein transport inhibitor GolgiStop (monensin, Cat. No. 554724, Becton-Dickinson, Franklin Lakes, N.J., USA) at 4 μl/6ml of culture medium.

Cells are washed cells twice in FACS staining buffer (PBS; 2 mM EDTA; 2% FBS), centrifuged at 1500 rpm for 5 min at either 4° C. or room temperature in an IEC Centra/GP8R large centrifuge with refrigeration (Thermo Fisher Scientific, Inc., Waltham, Mass., USA).

Staining is performed in 15 ml conical tubes. Cells are resuspended in 200 μl FACS staining buffer. Antibodies αCD3 PE-Cy7 (phycoerythrin-cyanine 7, e.g. Cat. No. 341091 or 557851) and αCD8-PB (pacific blue, Cat. No. 558207) (both BD Biosciences, San Jose, Calif., USA) are each added at 5 μl/10⁶ cells, and incubated 20 minutes at 4° C.

Cells are washed once in 3-5 ml staining buffer, centrifuged at 1500 rpm for 5 minutes at either 4° C. or room temperature, and aspirated.

Cells are fixed in cold Cytofix/Cytoperm solution (BD Biosciences Cat. No. 554722, or from kit 554714) added at 100 μl/10⁶ cells and brought to a final concentration of 20 mM ribonucleoside vanadyl complexes (RVC) (Cat. No. R3380) (Sigma, St. Louis, Mo., USA), e.g. using 0.1 volume of 200 mM RVC stock solution. In some embodiments, a final concentration of 10 mM RVC is used. Cytofix/Cytoperm solution comprises 4% paraformaldehyde with 0.1% saponin (a cell-permeabilizing agent). The resulting mixture is incubated on ice for 20 minutes.

Cells are washed twice in 3 ml cold 1× Perm/Wash buffer (BD Biosciences Cat. No. 554723), centrifuged at 1500 rpm for 5 minutes at 4° C., and aspirated. 1× Perm/Wash buffer comprises PBS with 0.1% saponin and 3% fetal calf serum (FCS), and is supplied commercially as a 10× concentrate.

Cells are then resuspended in 200 μl 1× Perm/Wash buffer.

### Sorting

For sorting cells based on intracellular levels of IL-17 and IFNγ, antibodies αhumanIL-17-AF647 (Alexa Fluor® 647, 20 μl/10⁶ cells) (Cat. 51-7179, eBioscience, Inc., San Diego, Calif., USA) and αhumanIFNγ-FITC (fluorescein isothiocyanate, 2 μl/10⁶ cells) (Cat. 554700, BD Biosciences, San Jose, Calif., USA) are added. No RVC is added at this step. The resulting mixture is incubated one hour at 4° C.

Cells are washed once in 3 ml cold 1× Perm/Wash buffer, centrifuged at 1500 rpm for 5 minutes at 4° C., and aspirated.

Cells are then resuspended in 1.5 ml FACS staining buffer containing 10 mM RVC in polystyrene 5 ml FACS tubes. In some embodiments, 5 mM RVC is used. Cells are passed through a cell strainer (BD Falcon Cell Strainer 100 μM, Cat. 352360, BD Biosciences, San Jose, Calif., USA) and kept on ice.

Cells are then sorted on a BD FACSAria™ II cell sorter (BD Biosciences, San Jose, Calif., USA) gated on CD3⁺ CD8⁻ cells. Cells are collected in 5 ml polypropylene FACS tubes containing 900 μl PBS and 100 μl 200 mM RVC, giving a final concentration of 20 mM RVC.

Sorted cells are then centrifuged at 1600 rpm for 5 min at 4° C. in an Eppendorf Centrifuge Model 5414 D. The supernatant is then discarded by inverting the tube, rather than aspiration, which can result in loss of cells. It is better to leave some liquid at this stage rather than aspirate and lose cells, particularly if cell number is expected to be low. Extra liquid is removed at the RNA extraction step anyway. The resulting cell pellet is then frozen. At least 50,000 cells are preferred for RNA extraction.

### RNA Extraction

RNA is extracted using the Roche High Pure FFPE RNA Micro Kit (Cat. No. 04 823 125 001) (Roche Applied Science, Indianapolis, Ind., USA) or the AMBION® Recoverall Total Nucleic Acid Isolation Kit for FFPE (Cat. No. AM1975) (Applied Biosystems, Foster City, Calif., USA), although the Ambion kit is less effective at extracting RNA from the low cell numbers.

Cell pellets are thawed on ice, centrifuged at 1550 rpm for 5 minutes at 4° C., and the supernatant liquid is aspirated. Sixty μl of Tissue Lysis Buffer and 10 μl 10% SDS buffer are added to each pellet, which are resuspended and transferred to 1.5 ml Eppendorf microcentrifuge tubes. The tubes are vortexed, spun down, and 30 μl Proteinase K working solution is added. Tubes are then vortexed and incubated at 55° C. for three hours.

Two hundred μl of Binding Buffer and 200 μl 100% ethanol (ACS grade minimum) are then added, and the tubes are vortexed and spun down. The lysates are then pipetted into the upper reservoir of a High Pure filter tube connected to a collection tube. The tubes are then centrifuged at 8,000×g for 30 seconds in a microcentrifuge and the flowthrough it discarded. Additional aliquots of lysate may be spun through the High Pure filter tube to load the column with additional sample material, albeit with care not to overload the column. The tube is then centrifuged one minute at maximum speed to dry the filter.

Thirty μl of DNase Solution, comprising 3 μl DNase Incubation Buffer and 27 μl DNase, is added and incubated for 15 minutes at room temperature. Three hundred μl of Wash Buffer I working solution is then added to the upper reservoir, and the tube is centrifuged at 8,000×g for 15 seconds. The flowthrough is discarded. Three hundred 300 μl of Wash Buffer II working solution is then added, and the tube is centrifuged at 8,000×g for 15 seconds, and again the flowthrough is discarded. Two hundred 200 μl of Wash Buffer II working solution is added, and the tube is centrifuged at 8,000×g for 15 seconds, and again the flowthrough is discarded as well as the collection tube.

The High Pure Micro filter tube is connected to a fresh collection tube and centrifuged at maximum speed for 2 minutes. The High Pure Micro filter tube is placed in a fresh 1.5 ml reaction tube, and 20 μl of Elution Buffer is added and incubated at room temperature for one minute. The tube is then centrifuged at 8,000×g for one minute. The eluate is then reloaded to the upper reservoir of the High Pure Micro filter tube, incubated at room temperature for another minute, and again centrifuged at 8,000×g for one minute.

The microcentrifuge tube, containing the eluted RNA, may be stored at −80° C. for later analysis. Gel electophoretic analysis of a sample RNA purified using the method of the present invention (“SPB protocol”), as compared to a typical prior art RNA preparation and purification method (Control“), is provided at FIG. 1.

### RT-qPCR Analysis of Gene Expression

RNA extracted as described above is then used in reverse-transcription quantitative polymerase chain reaction (RT-qPCR) experiments, as is known in the art, to evaluate gene expression in the sample from which the RNA was obtained. Briefly, RNA is reverse transcribed and cDNA is amplified using the Nugen WT-Ovation™ Pico RNA Amplification System (Nugen Technologies, San Carlos, Calif., USA). Real-time quantitative PCR is performed as previously described at Wilson et al. (2007) Nat. Immunol. 8:950, the disclosure of which is hereby incorporated by reference in its entirety. Briefly, real-time quantitative PCR is performed on 10 ng of cDNA from each sample using either of two methods. In the first method, two gene-specific unlabelled primers are used at 400 nM in an Applied Biosystems SYBR® green real-time quantitative PCR assay using an ABI 7000, 7300 or 7900 instrument. In the second method, two unlabelled primers at 900 nM each are used with 250 nM FAM-labeled probe (Applied Biosystems) in a TAQMAN™ real-time quantitative PCR reaction on an ABI 7000, 7300 or 7700 sequence detection system. Quantities of transcripts encoding ubiquitin (UBB) are measured in a separate reaction and used to normalize the data by the ΔCt method of Fehniger et al. (1999) J Immunol. 162:4511, the disclosure of which is hereby incorporated by reference in its entirety.

The primers presented at Table 1 are used (5′-3′) for detection of IL-17A, IFNγ, IL17F, IL22, CCL20 and IL26 transcripts in the sorted cells.

TABLE 1
PCR Primers

| | | | SEQ |
|---|---|---|---|
| Gene | Direction | Primer (5′→3′) | ID NO |
| IL17A | forward | CAACCGATCCACCTCACCTT | 1 |
| IL17A | reverse | GGCACTTTGCCTCCCAGAT | 2 |
| IFNγ | forward | CTTTAAAGATGACCAGAGCATCCA | 3 |
| IFNγ | reverse | ATCTCGTTTCTTTTTGTTGCTATTGA | 4 |
| IL17F | forward | TGCCAGGAGGTAGTATGAAGCTT | 5 |
| IL17F | reverse | ATGCAGCCCAAGTTCCTACACT | 6 |
| IL22 | forward | GCAGGCTTGACAAGTCCAACT | 7 |
| IL22 | reverse | GCCTCCTTAGCCAGCATGAA | 8 |
| CCL20 | forward | CTGGCTGCTTTGATGTCAGTG | 9 |
| CCL20 | reverse | GCAGTCAAAGTTGCTTGCTGC | 10 |
| IL26 | forward | TTTGAGGTGTGGGTTGCTGTTA | 11 |
| IL26 | reverse | TCAACAGCTTGGGACAATGTTC | 12 |

### Example 3

### Characterization of IL-17⁺, IFNγ³⁰ and IL17⁺/IFNγ⁺ and Th17 Cells

Buffy coats were obtained from normal healthy human volunteer blood donors. Naïve CD4⁺ CD45RO⁻ CD25⁻ T cells were isolated. See Wilson et al. (2007) Nat. Immunol. 8:950. T cells were activated using beads coated with anti-CD3/CD28/CD2 antibodies (1 bead : 10 cells). Th17 cells were polarized in the presence of 50 ng/ml human IL-23, 50 ng/ml hIL-1β (R&D Systems, Minneapolis, Minn., USA), and 10 μM PGE2 (Sigma, St. Louis, Mo., USA). Th1 cell differentiation was induced in the presence of 5 ng/ml hIL-12 (R&D Systems, Minneapolis, Minn., USA). After a 10-12 day culture period, CD4⁺ CCR6⁺ Th17 cells or Th1 cells were expanded seven days in the presence of 100 U/ml IL-2 (R&D Systems, Minneapolis, Minn., USA) and/or the indicated polarizing cytokines.

Cells were then stained for intracellular IL-17⁺ and IFNγ⁺, sorted, and subjected to gene expression analysis substantially as described in Example 2. Briefly, after Th17- or Th1-polarization and activation, CD3⁺ CD8⁻ T cells were sorted based on intracellular IL-17 and IFNγ content. FIGS. 2A and 2B. Real time PCR analysis of IL-17 and IFNγ expression for six different sort populations are shown at FIG. 3A.

Mean fluorescence intensity for IL-17 for IL17⁻/IFNγ⁻ or IL17⁻/IFNγ⁺ populations from both Th17 and Th1 polarized cells are provided at FIG. 3B. Lines connect Th17 and Th1 data for samples obtained from the same human subjects.

Real time PCR analysis of IL-17F, IL-22, CCL20 and IL-26 expression for the six different sort populations are shown at FIGS. 4A-4D, respectively.

What is claimed is:

1. A method of preparing mRNA from a preparation of cells comprising:
a) adding RVC to the preparation;
b) fixing the cells with formalin;
c) permeabilizing the cells with mild detergent; and
d) extracting mRNA in the presence of a de-crosslinking agent.

2. The method of claim 1 further comprising, between steps (c) and (d), a step for sorting the cells by fluorescence activated cell sorting.

3. The method of claim 2 wherein the RVC is added to a final concentration of about 1 to 20 mM during fixation.

4. The method of claim 3 wherein the RVC is added to a final concentration of about 2 to 5 mM during fixation.

5. The method of claim 2 wherein the mild detergent is saponin.

6. The method of claim 5 wherein the saponin is present at a concentration of 0.1%.

7. The method of claim 2 wherein the de-crosslinking agent is proteinase K.

8. The method of claim 2, further comprising resuspending the cells for intracellular immunostaining using a buffer that does not contain RVC, wherein the resuspending occurs after fixing and permeabilization, but prior to sorting.

9. A method of measuring gene expression in a population of cells sorted from a starting preparation of cells comprising:
a) adding RVC to the preparation of cells;
b) fixing the preparation of cells with formalin;
c) permeabilizing the preparation of cells with mild detergent;
d) sorting the preparation of cells by fluorescence activated cell sorting;
e) extracting mRNA in the presence of a de-crosslinking agent; and
f) measuring mRNA levels for one or more genes.

10. The method of claim 9 wherein mRNA is measured by quantitative RT-PCR.

11. The method of claim 10 wherein the RVC is added to a final concentration of about 1 to 20 mM.

12. The method of claim 11 wherein the RVC is added to a final concentration of about 2 to 5 mM.

13. The method of claim 10 wherein the mild detergent is saponin.

14. The method of claim 13 wherein the saponin is present at a concentration of 0.1%.

15. The method of claim 10 wherein the de-crosslinking agent is proteinase K.

16. The method of claim 10, further comprising resuspending the cells for intracellular immunostaining using a buffer that does not contain RVC, wherein the resuspending occurs after fixing and permeabilization, but prior to sorting.


SEQUENCE LISTING (12 sequences)

SEQ ID NO: 1 - DNA, 20 bp, Artificial
  PCR Primer
caaccgatccacctcacctt

SEQ ID NO: 2 - DNA, 19 bp, Artificial Sequence
  PCR primer
ggcactttgcctcccagat

SEQ ID NO: 3 - DNA, 24 bp, Artificial Sequence
  PCR primer
ctttaaagatgaccagagcatcca

SEQ ID NO: 4 - DNA, 26 bp, Artificial Sequence
  PCR primer
atctcgtttctttttgttgctattga

SEQ ID NO: 5 - DNA, 23 bp, Artificial Sequence
  PCR primer
tgccaggaggtagtatgaagctt

SEQ ID NO: 6 - DNA, 22 bp, Artificial Sequence
  PCR primer
atgcagcccaagttcctacact

SEQ ID NO: 7 - DNA, 21 bp, Artificial Sequence
  PCR primer
gcaggcttgacaagtccaact

SEQ ID NO: 8 - DNA, 20 bp, Artificial Sequence
  PCR primer
gcctccttagccagcatgaa

SEQ ID NO: 9 - DNA, 21 bp, Artificial Sequence
  PCR primer
ctggctgctttgatgtcagtg

SEQ ID NO: 10 - DNA, 21 bp, Artificial Sequence
  PCR primer
gcagtcaaagttgcttgctgc

SEQ ID NO: 11 - DNA, 22 bp, Artificial Sequence
  PCR primer
tttgaggtgtgggttgctgtta

SEQ ID NO: 12 - DNA, 22 bp, Artificial Sequence
  PCR primer
tcaacagcttgggacaatgttc